(19) United States
(12) Patent Application Publication
(10) Pub. No.: US 2004/0072351 A1
(43) Pub. Date: Apr. 15, 2004

(54) Methods and compositions for expressing a nucleic acid in a dendritic cell

(76) Inventors: Karl L. Womer, Gainesville, FL (US); Sihong Song, Gainesville, FL (US); Terence R. Flotte, Alachua, FL (US); Scott A. Loiler, Gainesville, FL (US); Mark A. Atkinson, Gainesville, FL (US); Michael Clare-Salzler, Gainesville, FL (US); Jude Samulski, Chapel Hill, NC (US); Chengwin Li, Carrboro, NC (US)

Correspondence Address: Stanley A. Kim, Ph.D., Esq., Akerman Senterfitt, Suite 400, 222 Lakeview Avenue, West Palm Beach, FL, 33402-3188, US

(21) Appl. No.: 10/427,165

(22) Filed: Apr. 30, 2003

Related U.S. Application Data
(60) Provisional application No. 60/376,595, filed on Apr. 30, 2002.

(51) Int. Cl. A61K 48/00; C12N 15/861

(52) U.S. Cl. 435/456; 424/93.2

(57) ABSTRACT

Recombinant adeno-associated virus (rAAV) virions having a serotype 1 capsid protein are effective for transferring heterologous nucleic acids into DC.

### CROSS-REFERENCE TO RELATED APPLICATIONS

The present application claims the priority of U.S. provisional patent application No. 60/376,595 filed on Apr. 30, 2002.

### FIELD OF THE INVENTION

The invention relates generally to the fields of molecular biology and gene delivery. More particularly, the invention relates to the use of recombinant adeno-associated virus (rAAV) vectors for expressing a nucleic acid in a dendritic cell (DC).

### BACKGROUND OF THE INVENTION

DCs are potent antigen presenting cells that play an important role in regulating immune responses (Steinman, Annu Rev Immunol., 9:271-96, 1991; Steinman and Nussenzweig, Proc Natl Acad Sci U S A. 99:351-358, 2002; Hart, Blood. 90:3245-3287, 1997; Banchereau, Annu Rev Immunol. 18:767-811, 2000; and Clare-Salzer, J Clin Invest., 90:741, 1992. Transfer of exogenous genes into DC can be used to modulate an immune response in a desired manner. A variety of strategies have utilized transgenic DCs to enhance a desired immune response as well as to inhibit an undesired one. E.g., Oberholzer et al., J Immunol., 168:3412, 2002.

Several different vectors have been used to introduce and express an exogenous nucleic acid in a eukaryotic cell. Those derived from naturally occurring viruses have proven to be particularly useful. Prominent examples of such vectors include those derived from adenovirus, adeno-associated virus (AAV), and various retroviruses. While each of these different vector types has proven capable of transferring exogenous genetic material into mammalian cells, none has proven to be ideal for all use with all cell types. For introducing an exogenous nucleic acid into a DC, a desirable vector is one that results in high expression of the exogenous nucleic acid, one that does not injure the host cell animal or induce an undesired immune response (e.g., against the vector itself), and one that does not alter the biology of the DC (e.g., induce maturation or alter functional aspects of the cell such as IL-12 production).

### SUMMARY

The invention relates to the discovery that some serotypes of rAAV are more effective than others for transferring genes into DC. In particular, the experiments described herein show that rAAV serotype 1 (rAAV1) was clearly superior to rAAV serotype 2 (rAAV2) at transferring genes into DC. In addition, rAAV serotypes 3, 4, and 5 (rAAV3, rAAV4, and rAAV5) were capable of transferring genes into DC.

Because rAAV1-mediated gene transfer into DC is so efficient and does not cause DC maturation, it is expected that the invention will be useful for, among other things, (1) developing products that alter DC to produce a protein useful for treating a disease involving a deficiency of that protein, (2) altering DC to induce an immune response against various cancers and pathogens, and (3) altering DC to prevent transplant rejection or autoimmune conditions. In addition, application of the method to immature DC may be particularly important in developing tolerance induction paradigms for transplantation and autoimmunity.

Accordingly, the invention features a method including the step of infecting a dendritic cell with a rAAV virion having at least one AAV serotype 1 capsid protein, or a rAAV virion having at least one AAV capsid protein from AAV serotype 3, 4, or 5. In the method, the rAAV virion can also include a non-AAV nucleic acid that can be interposed between a first AAV terminal repeat (TR) and a second AAV TR. The first and/or the AAV TR can be a serotype 2 TR. The non-AAV nucleic acid can be on that encodes a polypeptide. The step of infecting the dendritic cell with the rAAV virion can be one that results in expression of the polypeptide in the dendritic cell. The dendritic cell can be one in an in vitro cell culture, and/or one that is an immature dendritic cell. The invention also features a dendritic cell infected with a rAAV virion according to the above method.

Unless otherwise defined, all technical terms used herein have the same meaning as commonly understood by one of ordinary skill in the art to which this invention belongs. Commonly understood definitions of molecular biology terms can be found in Rieger et al., Glossary of Genetics: Classical and Molecular, 5th edition, Springer-Verlag: New York, 1991; and Lewin, Genes V, Oxford University Press: New York, 1994. Commonly understood definitions of virology terms can be found in Granoff and Webster, Encyclopedia of Virology, 2nd edition, Academic Press: San Diego, Calif., 1999; and Tidona and Darai, The Springer Index of Viruses, 1st edition, Springer-Verlag: New York, 2002. Commonly understood definitions of microbiology can be found in Singleton and Sainsbury, Dictionary of Microbiology and Molecular Biology, 3rd edition, John Wiley & Sons: New York, 2002.

By the term “gene” is meant a nucleic acid molecule that codes for a particular protein, or in certain cases a functional or structural RNA molecule.

As used herein, a “nucleic acid” or a “nucleic acid molecule” means a chain of two or more nucleotides such as RNA (ribonucleic acid) and DNA (deoxyribonucleic acid). A “non-AAV nucleic acid” is a nucleotide sequence not native to AAV.

As used herein, “protein” or “polypeptide” are used synonymously to mean any peptide-linked chain of amino acids, regardless of length or post-translational modification, e.g., glycosylation or phosphorylation.

As used herein, the term “vector” refers to a nucleic acid molecule capable of transporting another nucleic acid to which it has been linked. Vectors capable of directing the expression of genes to which they are operatively linked are referred to herein as “expression vectors.”

A first nucleic-acid sequence is “operably” linked with a second nucleic-acid sequence when the first nucleic-acid sequence is placed in a functional relationship with the second nucleic-acid sequence. For instance, a promoter is operably linked to a coding sequence if the promoter affects the transcription or expression of the coding sequence. Generally, operably linked nucleic acid sequences are contiguous and, where necessary to join two protein coding regions, in reading frame.

By the term “pseudotyped” is meant a nucleic acid or genome derived from a first AAV serotype that is encapsidated or packaged by an AAV capsid containing at least one AAV Cap protein of a second serotype. By “AAV inverted terminal repeats”, “AAV terminal repeats, “ITRs”, and “TRs” are meant those sequences required in cis for replication and packaging of the AAV virion including any fragments or derivatives of an ITR which retain activity of a full-length or WT ITR.

As used herein, the terms “rAAV vector” and “recombinant AAV vector” refer to a recombinant nucleic acid derived from an AAV serotype, including without limitation, AAV1, AAV2, AAV3, AAV4, AAV5, AAV6, AAV7, etc. rAAV vectors can have one or more of the AAV WT genes deleted in whole or in part, preferably the rep and/or cap genes, but retain functional flanking ITR sequences. A “recombinant AAV virion” or “rAAV virion” is defined herein as an infectious, replication-defective virus composed of an AAV protein shell encapsulating a heterologous nucleotide sequence that is flanked on both sides by AAV ITRs.

By the term “rAAV1” is meant a rAAV virion having at least one AAV serotype 1 capsid protein. Similarly, by the term “rAAV2” is meant a rAAV virion having at least one AAV serotype 2 capsid protein.

As used herein, the term “immature DC” refers to those DC that exhibit low level expression of costimulatory molecules, CD80/86, CD40; poor ability to induce T cell activation; inability to produce IL-12p70; and the potential to induce regulatory or anergic T cells.

By the phrase “DC activity” is meant any activity or function performed by a DC.

The term “infect” is used herein to signify the entry of a virion into a host cell regardless of whether or not the virion replicates in the host cell.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

FIG. 1 is a graph comparing human alpha-1-antitrypsin (hAAT) expression in DC transduced with rAAV vectors of serotypes 1-5.

FIG. 2 is a pair of graphs showing timing and dosing of rAAV1 infection. (A) Supernatant concentration of human AAT on day 10 of culture following infection of bone marrow cells (day 0), unstimulated DCs (day 4) or stimulated DCs (day 4) with 10³ multiplicity of infection (MOI) of rAAV serotype 1-human AAT. (B) Transgene expression by viral dose.

FIG. 3 is a pair of plots showing effects of rAAV1 infection/transduction on DC maturation by cell surface CD86 and major histocompatibility class (MHC) class II FACS staining. (A) Recombinant AAV1-human AAT-transduced DCs. (B) Nontransduced DCs.

FIG. 4 is a series of highly schematic illustrations showing rAAV vector constructs and transduction of mouse bone marrow-derived DCs with rAAV vectors. (A) Construction of individual rAAV serotype clones. (B) Diagram of the CB-AT vector construct. (C) Transduction of mouse bone marrow-derived DCs with rAAV virions.

### DETAILED DESCRIPTION

The invention provide methods and compositions for infecting DC with rAAV virions. Those rAAV virions with an AAV serotype 1 capsid infected DC much more effectively than rAAV virions lacking a serotype 1 capsid. The below described preferred embodiments illustrate adaptations of these compositions and methods. Nonetheless, from the description of these embodiments, other aspects of the invention can be made and/or practiced based on the description below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Conventional methods of gene transfer and gene delivery can also be adapted for use in the present invention. See, e.g., Gene delivery Methods: ed. M. I. Phillips, Vol. 436, Methods in Enzymology, Academic Press, 2002; Gene delivery: Principles and Applications, ed. T. Blackenstein, Springer Verlag, 1999; and Gene delivery Protocols (Methods in Molecular Medicine), ed. P. D. Robbins, Humana Press, 1997.

### Dendritic Cells

The invention provides compositions and methods for rAAV1-mediated expression of a non-AAV nucleic acid in a DC. DCs that might be used include mammalian DCs such as those from mice, rats, guinea pigs, non-human primates (e.g., chimpanzees and other apes and monkey species), cattle, sheep, pigs, goats, horses, dogs, cats, and humans. The DCs may be those within a mammalian subject (i.e., in vivo), or those within an in vitro culture (e.g., those cultured in vitro for ex vivo delivery to a subject). DCs according to the invention contain a nucleic acid including a non-AAV nucleic acid interposed between two AAV ITRs. In preferred DCs, the non-AAV nucleic acid is expressed, resulting in a polypeptide.

DCs can be obtained from any suitable source, including the skin, spleen, bone marrow, or other lymphoid organs, lymph nodes, or blood. Preferably, DCs are obtained from blood or bone marrow for use in the invention. Typically, DCs are generated from bone marrow and peripheral blood mononuclear cells (PBMC) after stimulation with exogenous granulocyte-macrophage colony stimulating factor (GM-CSF) and interleukin-4. Methods for obtaining DCs from bone marrow cells and culturing DCs are described in Inaba et al., J. Exp. Med. 176:1693-1702, 1992; and Bai et al., Int. J. Oncol. 20:247-253, 2002. Methods for culturing DCs from hematopoietic progenitor cells (Mollah et al., J. Invest. Dermatol. 120:256-265, 2003) and monocytes (Nouri-Shirazi and Guinet Transplantation 74:1035-1044, 2002) are also known in the art. An example of a large-scale monocyte-enrichment procedure for generating DCs is described in Pullarkat et al. (J. Immunol. Methods 267:173-183, 2002). DCs may be isolated from a heterogeneous cell sample using DC-specific markers in a fluorescence-activated cell sorting (FACS) analysis (Thomas and Lipsky J. Immunol. 153:4016-4028, 1994; Canque et al., Blood 88:4215-4228, 1996; Wang et al., Blood 95:2337-2345, 2000). Immature DC are characterized by low level expression of costimulatory molecules, CD80/86, CD40; poor ability to induce T cell activation; inability to produce IL-12p70; and the potential to induce regulatory or anergic T cells. In comparison, mature DC produce IL-12p70 and express high levels of MHC class II antigens, CD80/86, and CD40, IL-12p70 production. A population of cells containing DCs as well as isolated DCs may be cultured using any suitable in vitro culturing method that allows growth and proliferation of the DCs.

### rAAV Vectors and Virions

The invention provides compositions and methods for expressing a non-AAV nucleic acid in a DC and modulating DC activity involving rAAV vectors and virions. An rAAV vector used in methods of the invention is a recombinant nucleic acid sequence that includes those AAV sequences required in cis for replication and packaging (e.g., functional ITRs) of the DNA into a virion. Additionally, the rAAV vectors contain a non-AAV nucleic acid such as therapeutic or reporter gene. Useful rAAV vectors have one or more of the AAV WT genes deleted in whole or in part, but retain functional flanking ITR sequences. The AAV ITRs may be of any serotype, but in preferred methods, because they are relatively well characterized, the ITRs are derived from serotype 2. Methods for use of rAAV vectors are discussed, for example, in Tal, J., J. Biomed. Sci. 7:279-291, 2000 and Monahan and Samulski, Gene delivery 7:24-30, 2000.

An rAAV virion used in methods of the invention is an infectious virus particle containing a rAAV vector. The capsid proteins composing the exterior, non-nucleic acid portion of the virion are encoded by the AAV cap gene. The cap gene encodes three viral coat proteins, VP1, VP2 and VP3, which are required for virion assembly. The construction of rAAV virions has been described. See, e.g., U.S. Pat. Nos. 5,173,414, 5,139,941, 5,863,541, and 5,869,305, 6,057,152, 6,376,237; Rabinowitz et al., J. Virol. 76:791-801, 2002; and Bowles et al., J. Virol. 77:423-432, 2003.

Techniques involving AAV nucleic acids and virions of different serotypes are known in the art and are described in Halbert et al., J. Virol. 74:1524-1532, 2000; Halbert et al., J. Virol. 75:6615-6624, 2001; and Auricchio et al., Hum. Molec. Genet. 10:3075-3081, 2001. The rAAV vectors used in the invention may be derived from any of several AAV serotypes including 1, 2, 3, 4, 5, 6, and 7. Preferred rAAV vectors for use in the invention are derived from serotype 2 (or mutants thereof). Preferred AAV virions for use in the invention are derived from serotype 1 (or mutants thereof). Particular AAV vectors and AAV proteins of different serotypes are discussed in Chao et al., Mol. Ther. 2:619-623, 2000; Davidson et al., PNAS 97:3428-3432, 2000; and Xiao et al., J. Virol. 72:2224-2232, 1998.

### Non-AAV Nucleic Acids

Within the invention are compositions and methods for expressing a non-AAV nucleic acid in a DC. A non-AAV nucleic acid is a nucleic acid that is not native to AAV. Non-AAV nucleic acids according to the invention are typically interposed between first and second AAV ITRs. Any non-AAV nucleic acid (e.g., DNA, RNA) may be used in the invention. Examples of non-AAV nucleic acids include marker or reporter genes (e.g., genes encoding green fluorescent protein, β-galactosidase, luciferase), as well as therapeutic genes (e.g., tumor or pathogen antigen-encoding genes). Preferred non-AAV nucleic acids are those that, when expressed in the DC, encode a polypeptide that modulates DC activity. A number of non-AAV nucleic acids that modulate DC activity (e.g., increase or decrease DC activity) are known, including nucleic acids encoding Flt-3, GM-CSF, IL-3, G-CSF, TFG-beta, TNF-alpha, IL-10, IL-13, IL-4, IL-19, IL-21, IL-22, IL-23, interferon-gamma, and interferon-alpha inflammatory cytokines. Particularly preferred non-AAV nucleic acids are those that, when expressed in a DC, encode a polypeptide that modulates an immune response in a subject. For example, a DC containing a nucleic acid encoding a protein that induces tolerance in the subject is particularly useful. Additionally, a DC containing a nucleic acid encoding an antigen (e.g., a peptide fragment of a protein) that results in generation of an immune response directed against the antigen is useful. Examples of useful antigens peptides derived from insulin, thyroid antigens, transplantation antigens, tumor antigens, microbial antigens. In some applications, a non-AAV nucleic acid according to the invention may encode more than one protein (e.g., immunomodulatory proteins).

In some applications, the non-AAV nucleic acid is operably linked to one or more expression control sequences. Generally, operably linked nucleic acid sequences are contiguous and, where necessary to join two protein coding regions, in reading frame. Such control elements can include control sequences normally associated with the selected non-AAV nucleic acid (e.g., gene). Alternatively, heterologous control sequences can be employed. Examples of expression control sequences include promoters, insulators, silencers, enhancers, initiation sites, termination signals, and polyA tails.

To achieve appropriate levels of expression of the non-AAV nucleic acid, any of a number of promoters suitable for use in the selected subject may be employed. Suitable promoters are selected on the basis of the subject and the cell type into which DCs will be administered, as well as the non-AAV nucleic acid to which the promoter is operably linked. In some applications, constitutive promoters of different strengths can be used to express a protein(s) encoded by a non-AAV nucleic acid. Inducible promoters may also be used in compositions and methods of the invention. Promoters for use in the invention include non-viral and viral promoters. An example of a non-viral promoter that may be used is the chicken beta actin promoter. Examples of viral promoters include cytomegalovirus (CMV) immediate early promoter, simian virus 40 (SV40) late promoter, mouse mammary tumor virus (MMTV) promoter, a herpes simplex virus (HSV) promoter, a rous sarcoma virus (RSV) promoter, and Adenovirus EIA and major late (MLP) promoters. Synthetic and hybrid promoters may also find use herein.

### Modulating DC Activity

Compositions and methods of modulating DC activity are also within the invention. In a method of modulating DC activity, the DC is infected with a rAAV virion having at least one AAV capsid protein of serotype 1, 3, 4, or 5, and a non-AAV nucleic acid. Any rAAV virion containing at least one capsid protein from AAV serotypes 1, 3, 4, or 5 may be used to infect the DC. For example, the rAAV virion may contain capsid proteins only from serotype 1. Similarly, the rAAV virion may contain capsid proteins only from serotypes 3, 4, or 5. Alternatively, the rAAV virion may contain capsid proteins from more than one serotype (e.g., serotypes 1 and 3). Such mutant rAAV virions are described above and are provided for in methods of the invention.

DCs perform a variety of immunological activities (Caux et al., Springer Semin. Immunopathology 22:345-369, 2000; Lane and Brocker Curr. Opin. Immunol. 11:308-313, 1999; and Morse et al., Expert. Opin. Biol. Ther. 2:35-43, 2002). These activities include recognition of foreign pathogens in tissues, upregulation of MHC, and expression of accessory and activation molecules such as CD80/B7-1, CD86/B7-1, CD54/immune cell adhesion molecule-1 (ICAM-1), and CD25/interleukin-2 (IL-2) receptor. Additional activities of DCs include processing and presenting antigen to antigen-specific, syngeneic T lymphocytes, stimulating proliferation of allogeneic T lymphocytes, and conjugating to T lymphocytes, particularly memory T lymphocytes. Methods of modulating DC activity according to the invention may involve modulating any of the above activities, for example, as well as any other activity performed by a DC.

In methods of the invention, expression of the non-AAV nucleic acid results in modulation of DC activity. Modulation of DC activity includes increasing and decreasing a WT DC activity, as well as inducing the DC to perform an activity not performed by WT DC. Any non-AAV nucleic acid that, when expressed in a DC, modulates activity of the DC may be used in the invention.

To modulate DC activity, any suitable method of infecting the DC with a rAAV virion may be used. For example, if the DC is within an in vitro culture of bone marrow cells, rAAV virions are added to the culture of bone marrow cells at a suitable MOI (e.g., 10¹-10³ MOI) and the culture is incubated under conditions that allow for infection of the DCs with rAAV. Methods for infecting DC with rAAV are described in Ponnazhagan et al., J. Virol. 75:9493-9501, 2001; Liu et al., J. Interferon Cytokine Res. 20:21-30, 2000; Zhang et al., J. Virol. 74:8003-8010, 2000; Liu et al., Cancer Gene Ther. 8:948-957, 2001; and Chiriva-Internati et al., Eur. J. Immunol. 32:30-38, 2002.

### Delivering rAAV To Cells and Subjects

Delivery of the rAAV virions of the invention to DC may be performed by simply infecting the DC with the virion. During the infecting step, a DC may be one within a subject or one in an in vitro cell culture (e g., one that is cultured in vitro for use in ex vivo delivery to a subject). To infect a DC with a rAAV virion in vivo, any suitable method for administering a rAAV virion to a mammalian subject may be used. For example, the rAAV may be administered to the subject by parenteral administration (e.g., intravenous injection). Alternatively, a DC of a subject may be infected with a rAAV virion using an ex vivo protocol. In an ex vivo protocol, the DC is first removed from the subject. DCs according to the invention can be removed from any suitable site in the subject, most preferably bone marrow. Bone marrow harvesting techniques are well known in the art (Inaba et al., J. Exp. Med. 176:1693-1702, 1992). The harvested DC is then cultured under conditions that allow proliferation of the cell. See, e.g., Id. The cultured DCs are then infected with at least one rAAV virion. In a preferred method of infecting a DC with a rAAV virion, the DC is cultured in vitro in the presence of at least one infectious rAAV virion under conditions that allow infection of the DC with rAAV. Methods for infecting cells with rAAV are described above. After infecting DCs with rAAV virions, the DCs are administered to the subject. Numerous methods of administering cells are known. See, e.g., Van Tendeloo et al., Leukemia 15:545-558, 2001; Marovich et al., J. Infect. Dis. 186:1242-1252, 2002; Paul et al., Curr. Gene Ther. 2:91-100, 2002; and Valone et al., Cancer J. suppl. 2:S53-61, 2001.

### Modulating an Immune Response in a Subject

The invention also provides methods for modulating an immune response in a subject. In these methods, a DC is infected with a rAAV virion containing at least one AAV capsid protein derived from AAV serotype 1, 3, 4, or 5 and a non-AAV nucleic acid. Any non-AAV nucleic acid that, when expressed in a cell of the subject, results in modulation of the immune response in a subject may be used in the invention. Preferred non-AAV nucleic acids encode a polypeptide that is expressed in the DC. Expression of a non-AAV nucleic acid that results in modulation of the immune response may result in an increase or decrease of the immune response in the subject. Examples of non-AAV nucleic acids that increase an immune response in a mammalian subject include TNF-alpha, interferon-alpha, IL-1beta, and IL-6. These immune response regulators can be expressed alone, but are preferably expressed in a combination in order to get the maximal desired effect. Examples of non-AAV nucleic acids that decrease an immune response in a mammalian subject include IL-10, IL-11, IL-13, IL19, and TGF-beta.

Compositions and methods for increasing and decreasing an immune response in a subject may be used in a variety of DC-based immunotherapy strategies for treating a variety of disorders. Mature DC are the key antigen presenting cell population which efficiently mediates antigen transport to organized lymphoid tissues for the initiation of T cell responses (e.g., induction of cytotoxic T lymphoctyes). The normal function of DCs is to present antigens to T cells, which then specifically recognize and ultimately eliminate the antigen source. DCs are used as both therapeutic and prophylactic vaccines for cancers and infectious diseases. Such vaccines are designed to elicit a strong cellular immune response. DC biology, gene transfer into DC, and DC immunotherapy are reviewed in Lundqvist and Pisa, Med. Oncol. 19:197-211, 2002; Herrera and Perez-Oteyza, Rev. Clin. Esp. 202:552-554, 2002; and Onaitis et al., Surg. Oncol. Clin. N. Am. 11:645-660, 2002.

The induction of cytotoxic and type 1 helper (Th1) cellular responses is highly desirable for vaccines targeting chronic infectious diseases or cancers (P. Moingeon, J. Biotechnol. 98:189-198, 2002). The use of rAAV-infected DCs expressing interleukins (e.g., IL-2, IL-12 and gamma interferon) that upregulate Th1 cells and their actions may be used to increase resistance to pathogens (J. W. Hadden, Int. J. Immunopharmacol. 16:703-710, 1994). For the treatment of HIV infection, for example, DCs can be targeted both ex vivo and in vivo to initiate and enhance HIV-specific immunity (Piguet and Blauvelt J. Invest. Dermatol. 119:365-369, 2002).

In addition to HIV therapies, DCs infected with rAAV may be used in cancer immunotherapies. DCs manipulated (e.g., infected with rAAV) to present tumor antigen to secondary lymphoid organs and resting, naive T-cells are useful for generating tumor-specific T-cells (A. F. Ochsenbein Cancer Gene Ther. 9:1043-1055, 2002). For example, DCs infected with rAAV encoding a myeloma-associated antigen may be useful as an anticancer therapy for multiple myeloma (Buehler and Hajek Med. Oncol. 19:213-218, 2002). DCs expressing certain cytokines or chemokines have been shown to display a substantially improved maturation status, capacity to migrate to secondary lymphoid organs in vivo, and ability to stimulate tumor-specific T-cell responses and induce tumor immunity in vivo. DCs infected with rAAV expressing cytokines, therefore, may be useful for inducing tumor immunity. The therapeutic role of DCs in cancer immunotherapy is reviewed in Lemoli et al., Haematologica 87:62-66, 2002; A. F. Ochsenbein, Cancer Gene Ther. 9:1043-1055, 2002; Zhang et al., Biother. Radiopharn. 17:601-619, 2002; Di Nicola et al., Cytokines Cell Mol. Ther. 4:265-273, 1998; D. Avigan, Blood Rev. 13:51-64, 1999, and Syme et al., J. Hematother. Stem Cell Res. 10:601-608, 2001. rAAV virions for expressing TNF-alpha, interferon alpha, IL-12p70, and IL-1beta may be used to mature and activate DC.

In an example of a rAAV/DC-based vaccine strategy, rAAV virions containing rAAV vectors (e.g., AAV2 vector pseudotyped in rAAV1 particles) encoding an immunogen are used to infect DCs, resulting in expression and presentation of the immunogen to resting, naive T-cells. Such an antigen presentation strategy can be used alone or in association, as part of mixed immunization regimens, in order to elicit broad immune responses. Different strategies of immunization involving delivery of DCs to patients are described in Onaitis et al., Surg. Oncol. Clin. N. Am. 11:645-660, 2002.

rAAV-infected DCs may also be used to modulate T-cell (Th1 and type 2 or Th2) responses for the treatment of autoimmune disorders (e.g., arthritis, asthma, atopic dermatitis). The balance between Th1 and Th2 cells is of importance in many autoimnimune disorders. Th1 cell activity predominates in joints of patients with rheumatoid arthritis and insulin-dependent diabetes mellitus, whereas Th2 cell-dominated responses are involved in the pathogenesis of atopic disorders (e.g., allergies), organ-specific autoimmune disorders (type 1 diabetes and thyroid disease), Crohn's disease, allograft rejection (e.g., acute kidney allograft rejection), and some unexplained recurrent abortions (Allergy Asthma Immunol. 85:9-18, 2000). Allograft rejection occurs when the host immune system detects samespecies, non-self antigens. To prevent or treat allograft rejection, DCs containing rAAV may be used to induce tolerance to tissue-specific antigens (B. Arnold Transpl. Immunol. 10:109-114, 2002). DC expressing immunosuppressive molecules may also be used as a therapy for allograft rejection (Lu and Thomson Transplantation 73:S19-22, 2002).

rAAV-infected DCs may further be used to induce an immune response against a microbial pathogen (e.g., viruses, bacteria, fungi, protozoa, and helminths). For example, the non-AAV nucleic acid of the rAAV virion might encode a peptide antigens derived from the microbial pathogen. Presentation of the antigen by DC infected with the virion could stimulate a vigorous immune response against the pathogen.

### Administration of Compositions

Ex vivo delivery of cells infected with rAAV virions is provided for within the invention. DC-mediated ex vivo gene delivery may be used to transplant rAAV-infected host DC back into the host. A suitable ex vivo protocol may include several steps. For example, a DC is first isolated from the host and the infected with an rAAV virion having a non-AAV nucleic acid. These genetically modified cells are then be transplanted back into the host. Several approaches may be used for the reintroduction of DCs into the host, including subcutaneous, intradermal, intravenous, and intrathynic administration. Autologous and allogeneic cell transplantation may be used according to the invention. Different strategies of delivering DCs to humans are described in Onaitis et al., Surg. Oncol. Clin. N. Am. 11:645-660, 2002.

### Effective Doses

The rAAV-infected cells described above are preferably administered to a mammal in an effective amount, that is, an amount capable of producing a desirable result in a treated subject (e.g., modulating DC activity and modulating an immune response in the subject). Such a therapeutically effective amount can be determined as described below.

Toxicity and therapeutic efficacy of the compositions utilized in methods of the invention can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration utilized.

As is well known in the medical and veterinary arts, dosage for any one animal depends on many factors, including the subject's size, body surface area, age, the particular composition to be administered, time and route of administration, general health, and other drugs being administered concurrently. It is expected that an appropriate dosage for intravenous administration of the compositions would be in the range of about 50,000 to 50,000,000 DC per animal. Additional doses could be administered to enhance or prolong the response.

### Examples

The present invention is further illustrated by the following specific examples, which should not be construed as limiting the scope or content of the invention in any way.

Example 1—Materials and Methods

Mice: Specific pathogen-free male C57BL/6 mice between five and eight weeks of age were maintained on standard rodent food and water ad libitum.

Generation of bone marrow-derived DCs: DCs derived from mouse bone marrow were generated as previously described (Inaba et al., J Exp Med. 176:1693-1702, 1992). Briefly, bone marrow cells harvested from the femur and tibia of C57BL/6 male mice were depleted of red blood cells by Iysis with 0.84% ammonium chloride. Thereafter, 10⁶ cells were cultured on 24 well plates (Costar, Corning, N.Y.) in RPMI 1640 (Cellgro, Hemndon, Va.) with 10% heat inactivated fetal calf serum, 0.000375% 2-mercaptoethanol (Sigma Chemical Co., St Louis, Mo.) and 1% penicillin-streptomycin-neomycin (Gibco, Grand Island, N.Y.), pH 7.2-7.4, supplemented with 500 units/mL of recombinant mouse GM-CSF (R&D Systems, Minneapolis, Minn.) and 1000 units/mL of recombinant mouse interleukin-4 (IL-4) (BD/PharMingen, San Diego, Calif.) and incubated in a 5% CO₂ atmosphere at 37° C. for up to 10 days. The medium was supplemented every 48 hours of culture with additional recombinant cytokines. These conditions yield a population of cells consisting of 40-60% DCs, with 20-40% of these DCs expressing a mature phenotype (see Antibodies and FACS analysis below). In certain groups, lipopolysaccharide (LPS) was added to culture on day 3, which induces 40-60% of the DC population to express a mature phenotype.

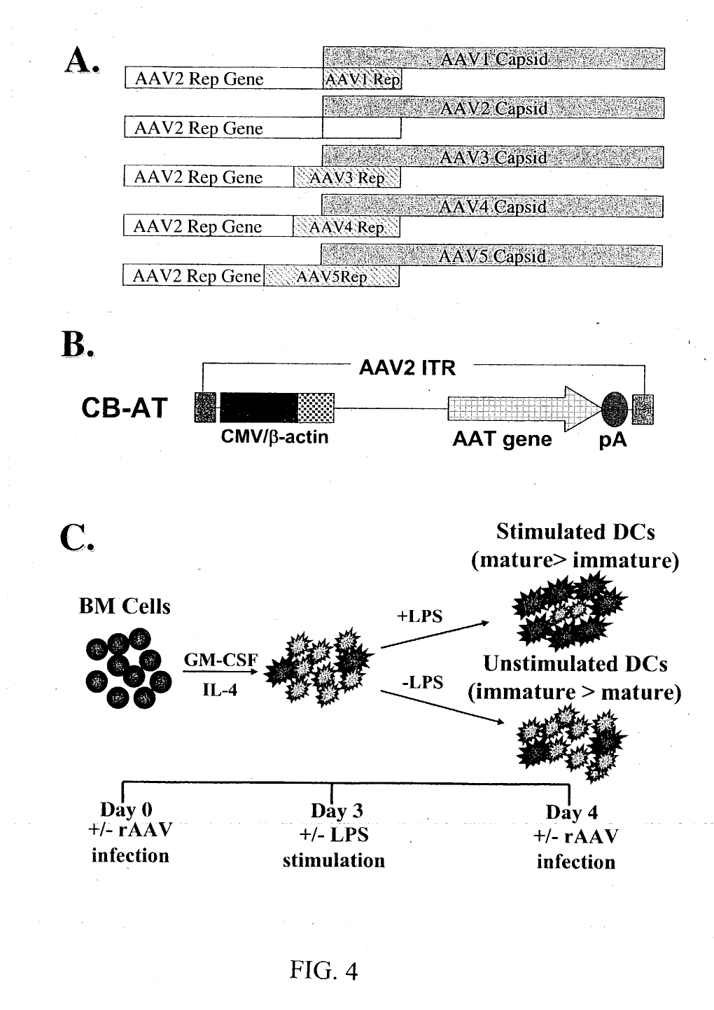

Plasmid construction, viral packaging and purification: Hybrid virions of AAV serotypes 1-5 containing the AAV2rep plasmid backbone/human AAT transgene were constructed as previously described (Rabinowitz et al., J Virol. 76:791-801, 2002). Briefly, each serotype-specific capsid domain (FIG. 4A, shaded rectangles) was cloned into the pBS+AAV2rep plasmid, with modifications containing the coding region of the carboxy termini of each serotype's rep coding domain (FIG. 4A, hatched rectangles) cloned into the constructs as needed. The CB-AT vector construct has been previously described (Song et al., Gene Ther. 8: 1299-1306, 2001 and Xu et al., Hum Gene Ther. 12:563-573, 2001) and is depicted in FIG. 4B. Briefly, it contains AAV2 ITRs and human AAT CDNA driven by a CMV enhancer and CB promoter. A bovine growth hormone pA is included. Standard transfection protocols (Rabinowitz et al., Virology. 265:274-285, 1999) were used with modifications as previously described (Rabinowitz et al., J Virol. 76:791-801, 2002). All five serotypes of rAAV virions were produced, purified, and titered as previously described (Chao et al., Mol Ther. 2:619-623, 2000 and Chao et al., Gene Ther. 6:1695-1704, 1999).

rAAV transduction of mouse bone marrow-derived DCs: Mouse bone marrow cells were cultured for 3 days in GM-CSF and IL-4 followed by LPS stimulation (1 μg/mL) on day 3 (stimulated DCs) or no stimulation (unstimulated DCs) as depicted in FIG. 4C. Cells were infected at each stage of differentiation/maturation (day 0 for bone marrow cells, day 4 for unstimulated or stimulated DCs) with rAAV at 10¹−10³ multiplicities of infection (MOI), depending upon the experiment. Negative control wells included cells not infected with virus. For experiments directly comparing the transduction efficiency of infection at different DC maturational stages, media was replaced on day 4 in all 3 groups to remove the human AAT that had accumulated in the bone marrow cell group (day 0 infection). Parallel experiments were performed using a coinfection of adenovirus serotype 5 at 5 plaque forming units (PFU) per cell for 2 hours at 37° C. prior to introduction of rAAV. C12 cells (HeLa-derived cell line), human aortic endothelial, and smooth muscle cells (Clonetics; Walkersville, Md.) infected with the same preparation of rAAV1-5 virions served as controls.

Detection of transgene expression: culture supernatants were collected at various time points, depending upon the experiment, and tested for human AAT expression using a modified double antibody-sandwich ELISA (Jooss et al., J Virol. 72:4212-4223, 1998 and Song et al., Gene Ther. 8: 1299-1306, 2001). In some experiments, cell populations were enriched for CD11c+ cells by positive bead selection (Miltenyi Biotec, Inc.), which consistently yields a population of >90-95% CD11c+ cells by FACS staining. Culture supernatants from these experiments were collected six days after CD11c+ enrichment and analyzed by ELISA.

Antibodies and FACS analysis: Cells were washed twice with FACS buffer: 1% bovine serum albumin (BSA), 1 mM EDTA (Fisher Scientific, Atlanta, Ga.) and 0.1% sodium azide (Sigma Chemical Co., St Louis, Mo.) in Hank's balanced solution without phenol red, calcium or magnesium (Cellgro, Herndon, Va.), followed by resuspension in 4% BSA FACS buffer. After blocking with CD16/CD32 Fc (Pharmingen, San Diego, Calif.), antibodies were used for staining, as described below. DCs were identified using antibodies against CD11c, whereas DC maturational status was assessed by staining with antibodies against CD86 and MHC class II (I-A^{d}/I-E^{d}) (Immunotech, Marseille, France). Antibodies were either directly conjugated to FITC, PerCp or PE (Pharmingen, San Diego, Calif.) or indirectly conjugated to an anti-biotin antibody labeled with APC (Molecular Probes, Eugene, Oreg.). Samples were analyzed by FACSCalibur, using CellQuest software (Becton Dickinson Systems, San Jose, Calif.). Analysis was based on 2×10⁴ live cells, as determined by the absence of 7-amino-actinomycin D (7-AAD) staining.

Statistical analysis: Data are presented as the mean ±SEM. Student's t test and ANOVA testing were used for statistical analyses comparing the different groups, with statistical significance considered if P<0.05.

### Example 2—Results

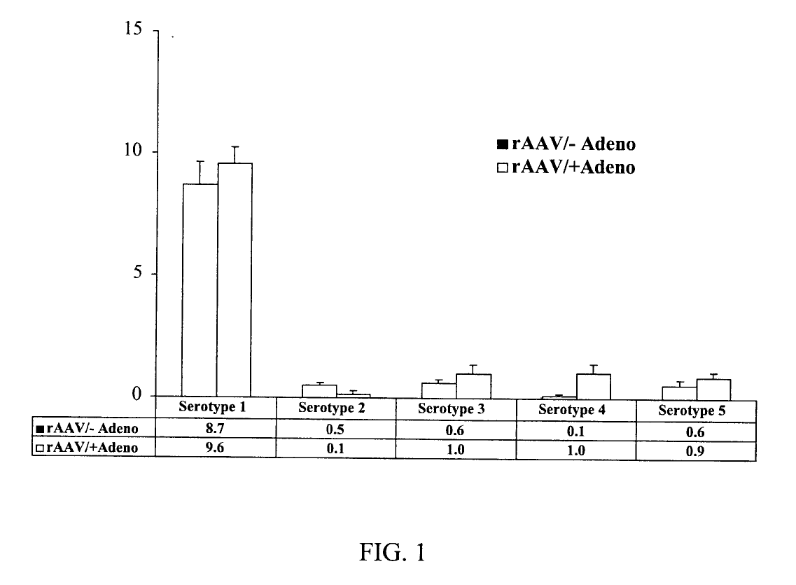

Mouse DC Cultures Are Transduced More Efficiently by rAAV Serotype 1: Unstimulated DCs were infected on day 4 of culture at 10³ MOI with the panel of hybrid rAAV serotype 1-5 vectors containing the human AAT transgene. As shown in FIG. 1, infection with rAAV serotype 1 resulted in significantly higher expression of human AAT in day 10 culture supernatants compared with any of the other four serotypes (P<0.0001, ANOVA). Unstimulated DCs were infected at a MOI of 10³ with the panel of hybrid rAAV serotype 1-5 virions containing the human AAT transgene. Parallel experiments were performed using a coinfection of adenovirus serotype 5 at 5 PFU per cell. AAT concentration in ng/ml is represented by the Y axis of the graph. Furthermore, coinfection with adenovirus had no significant effect on gene expression. Positive control experiments using C12 and human aortic endothelial and smooth muscle cell lines demonstrated successful transduction and gene expression of all serotypes, confirming that the superiority of rAAV serotype 1 on the DCs was not due to any loss of infectivity of the other preparations.

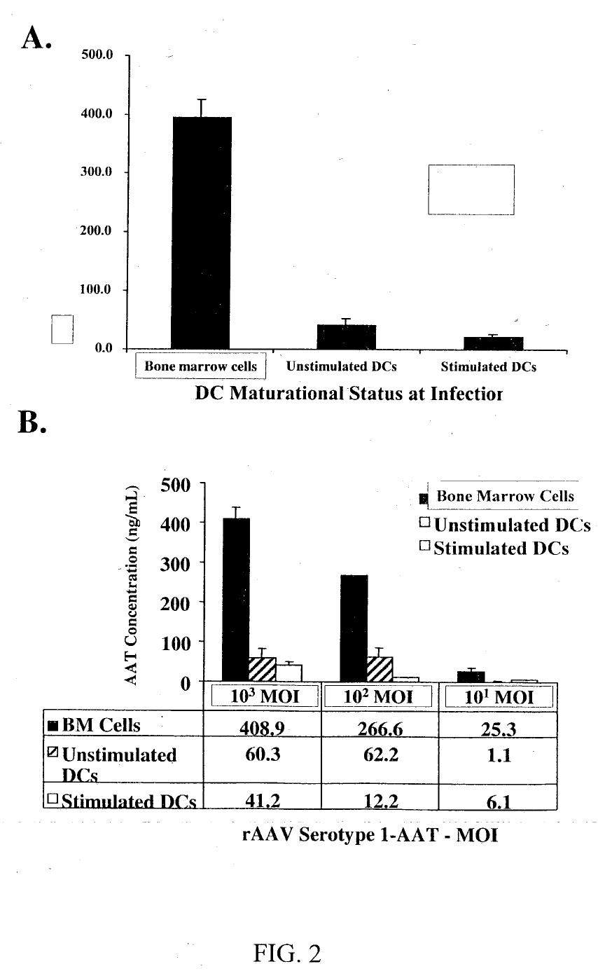

Gene Transfer at the Bone Marrow Cell Stage of DC Culture is Superior to Later Time Points: Additional studies were performed to determine the optimal rAAV1 dose and timing of infection during mouse myeloid DC differentiation and maturation. To allow for direct comparison of infectious efficacy, culture media was changed in all groups on day 4, thereby removing any accumulation of human AAT in the bone marrow cell group. As shown in FIG. 2A, infection with rAAV serotype 1 on day 0 of culture (bone marrow cells) resulted in considerably higher expression of human AAT by day 10 than infection on day 4 of culture (unstimulated>stimulated). Moreover, the human AAT concentration at day 4 in the bone marrow cell infection group (131.2±9.1 ng/mL) was higher than in the unstimulated or stimulated DC groups by day 10, suggesting superior transduction efficiency with infection of bone marrow cells as an explanation rather than a delay in genomic integration and gene expression with infection of established DCs at day 4. As shown in FIG. 2B, infection with rAAV1 exhibited a dose-dependent expression of the transgene. These results confirm the superiority of gene transfer at the bone marrow cell stage of DC culture, with as little as 10¹ MOI necessary for detectable gene expression in this group by day 10.

Transgene Expression Occurs After CD11c+ Enrichment: Although the mouse myeloid DC culture scheme described above typically yields between 40-60% DCs, as determined by cell surface CD11c staining, it is possible that other cell types were infected and therefore responsible for the human AAT expression measured in culture supernatants. Therefore, experiments were performed where bone marrow cells were infected with rAAV1 (10² MOI) and enriched for CD11c+ cells on day 4 of culture, which yields >90-95% CD11c+ cells by FACS analysis. These cells were then placed back in culture and supernatants tested 6 days later for human AAT expression. Results demonstrated human AAT expression (447.1±19.1 ng/mL) comparable to experiments using non-enriched populations. These results confirm that rAAV serotype 1 infects DCs.

Recombinant AAV Serotype 1 Transduction Does Not Induce DC Maturation:

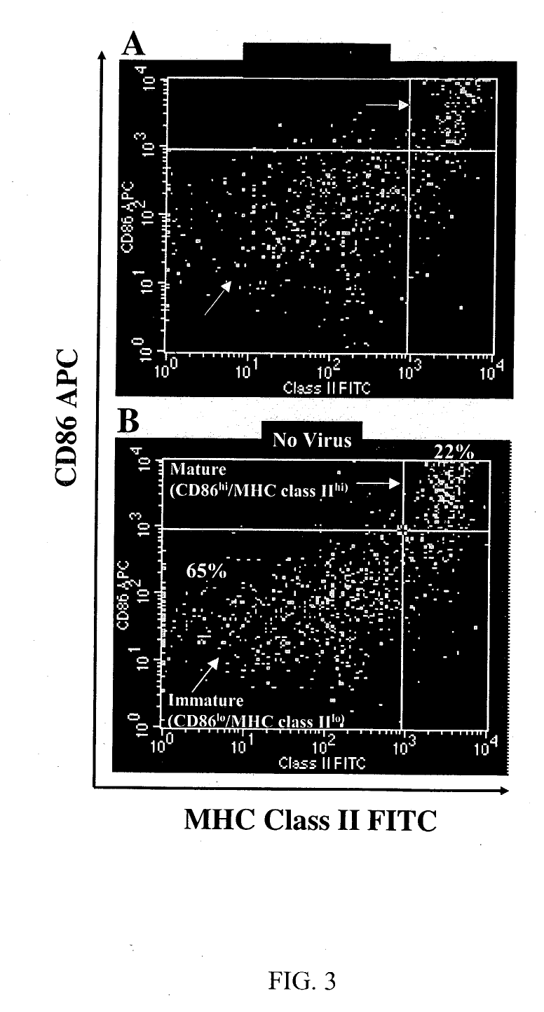

Studies using adenovirus vectors for gene transfer (Oberholzer et al., J Immunol. 168:3412-3418, 2002 and Korst et al., Mol Ther. 5:307-315, 2002) have shown virus-induced DC maturation. To determine whether similar effects occur following DC transduction with rAAV1 vectors, a FACS analysis for cell surface markers of DC maturation (CD86/MHC class II) was performed on day 6 of culture after infection of bone marrow cells with rAAV1. As shown in FIG. 3, rAAV1 infection failed to induce DC maturation over that seen in parallel DC cultures not infected with virus. These results demonstrate that rAAV1, unlike adenovirus, does not effect phenotypic maturation of DCs.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method comprising a step of infecting a dendritic cell with a rAAV virion comprising at least one AAV serotype 1 capsid protein.

2. The method of claim 1, wherein the rAAV virion further comprises a non-AAV nucleic acid.

3. The method of claim 2, wherein the non-AAV nucleic acid is interposed between a first AAV TR and a second AAV TR.

4. The method of claim 3, wherein the first AAV TR is a serotype 2 TR.

5. The method of claim 4, wherein the second AAV TR is a serotype 2 TR.

6. The method of claim 2, wherein the non-AAV nucleic acid encodes a polypeptide.

7. The method of claim 6, wherein the step of infecting the dendritic cell with the rAAV virion results in expression of the polypeptide in the dendritic cell.

8. The method of claim 1, wherein the dendritic cell is in an in vitro cell culture.

9. The method of claim 1, wherein the dendritic cell is an immature dendritic cell.

10. A method comprising the step of infecting a dendritic cell with a rAAV virion comprising at least one AAV capsid protein, wherein the capsid protein is of an AAV serotype selected from the group consisting of 3, 4, and 5.

11. The method of claim 10, wherein the rAAV virion further comprises a non-AAV nucleic acid.

12. The method of claim 11, wherein the non-AAV nucleic acid is interposed between a first AAV TR and a second AAV TR.

13. The method of claim 12, wherein the first AAV TR is a serotype 2 TR.

14. The method of claim 13, wherein the second AAV TR is a serotype 2 TR.

15. The method of claim 11, wherein the non-AAV nucleic acid encodes a polypeptide.

16. The method of claim 15, wherein the step of infecting the dendritic cell with the rAAV virion results in expression of the polypeptide in the dendritic cell.

17. The method of claim 10, wherein the dendritic cell is in an in vitro cell culture.

18. The method of claim 10, wherein the dendritic cell is an immature dendritic cell.

19. A dendritic cell infected with a rAAV virion according to the method of claim 1.

20. A dendritic cell infected with a rAAV virion according to the method of claim 10.
